(19) United States
(12) Patent Application Publication
(10) Pub. No.: US 2002/0187479 A1
(43) Pub. Date: Dec. 12, 2002

(54) In vitro capture of nucleic acids via modified oligonucleotides and magnetic beads

(76) Inventors: Gayle Dace, San Diego, CA (US); William Kimmerly, Agoura Hills, CA (US)

Correspondence Address: TORREY MESA RESEARCH INSTITUTE, INTELLECTUAL PROPERTY DEPARTMENT, 3115 MERRYFIELD ROW, SAN DIEGO, CA, 92121, US

(21) Appl. No.: 09/879,279

(22) Filed: Jun. 12, 2001

(51) Int. Cl. C12Q 1/68

(52) U.S. Cl. 435/6

(57) ABSTRACT

A method is disclosed for capturing one or more target simple sequence repeats (SSRs) utilizing locked nucleic acids and strand displacement stategies. In this method, one or more modified oligonucleotide conjugates are selected in which the conjugates are constructed from at least one locked nucleic acid (“LNA”) and a linking molecule, hereinafter referred to as “LNA conjugates”. When incubated with a sample of nucleic acids, these LNA conjugates capture SSRs by selectively binding to complementary sequences of DNA or RNA, which may be either single or double stranded. Once bound, the resulting complexes constitute hybridized duplexes containing both a targeted simple sequence repeat portion and a LNA conjugate portion. The captured SSRs are separated from the sample by using a linking source that binds to the linking molecule of the hybridized duplex and extracting the linking source with the bound duplex from the sample. In a preferred embodiment, the SSRs may be detached from the LNA conjugates by treatment with an alkaline buffer.

### FIELD OF THE INVENTION

The present invention relates to the field of DNA purification and preparation. More particularly, the present invention relates to products and methods of isolating simple sequence repeats utilizing strand displacement mechanisms.

### BACKGROUND OF THE INVENTION

The ability to isolate, clone, and synthesize nucleotide sequences has led to the development of a number of highly effective techniques in disease diagnosis, genetic analysis and the design of genetic therapeutics. Particularly, the detection of DNA or RNA sequences associated with a particular antigen, phenotype or genotype currently enjoys widespread use in modem medicine and scientific research.

Of particular interest is the isolation of simple sequence repeats (SSRs), which contain dinucleotide repeats, (e.g., (dT-dC)ₙ(dG-dA)ₙ) of a genome. Often the SSRs are hyper-variable in the number of repeat units (n) from individual to individual, or from line to line in inbred crops. They are highly informative DNA markers for genetic linkage mapping, marker-assisted breeding, genotyping, and trait ID. Unfortunately, SSRs have a low frequency of occurrence in most genomes (1 in every 100 kb or more), so an accurate and efficient method of detection and isolation is needed to identify genomic SSRs.

Typically, isolation of DNA, RNA, or other nucleotide sequences generally involves hybridization between a probe and a complementary target nucleotide sequence in order to isolate, identify, and analyze particular nucleotide sequences. Conventional techniques include either hybridization with a sample or probe nucleotide sequence immobilized onto a solid support, or hybridization of sample and probe sequences in solution, followed by separation of hybridized and unhybridized species. See, generally, Meinkoth et al. Anal. Biochem 138:267-284 (1984).

Unfortunately, immobilizing probes onto solid supports suffer from a number of substantial deficiencies. For example, many support materials such as agarose and cellulose generally have a low surface to volume ratio, resulting in poor hybridization kinetics, thereby reducing the sensitivity of the hybridization assay as well as significantly increasing the amount of time required to carry out the technique.

Alternatively, colony or plaque hybridization are well established methods, but they too have practical disadvantages. First, they require time-consuming and labor intensive steps of filter preparation that often limit the number of clones that can be screened. Second, since these procedures include prior denaturation steps and other treatments that destroy the integrity of the target DNA molecules, the corresponding clones must be isolated again from the original plates to obtain intact DNA molecules for further biological and biochemical manipulations. Frequently, many of these techniques entail a number or washing steps or other manipulations to decrease the amount of nonspecific nucleotide binding, thus requiring expensive, and possibly error inducing, skilled, human interactions.

In attempts to overcome these deficiencies capture by triplex forming oligonucleotides has been used to detect and isolate intact nucleotide sequences from double stranded DNA without prior denaturation. In this method, a single stranded oligonucleotide probe is complexed to double stranded DNA to form an intermolecular triple helix. Formation of the oligonucleotide probe has been described in U.S. Pat. Nos. 5,591,841 and 5,482,836, which are incorporated by reference herein in their entirety, including any figures. Typically, an oligonucleotide template is coupled to biotin to construct the oligonucleotide probe. Upon formation of a triple helix with complementary double stranded DNA, the helix is isolated using streptavidin-coated beads. The use of streptavidin coated beads as a separation mechanism is well documented.

While triple affinity capture has been used to isolate nucleotide sequences from double strand DNA, this method is limited by the absolute necessity of homopurine and homopyrimidine stretches of DNA residue in order to form the triple helix. The triplex structure is formed when pyrimidine thymine (T) recognizes adenine-thymine (AT) base pairs to form T-AT triplets. Likewise, pyrimidine cytosine (C) can recognize guanine-cytosine (GC) base pairs to form C-GC triplets. As such, nucleotide sequences that do not include homopurine or homopyrimidine rich residues can not be isolated using this method.

Recently, oligonucleotide analogues have been developed to investigate the conformational transition that occurs when oligonucleotides hybridize to a target sequence, from the relatively random coil structure of the single stranded state to the ordered structure of the duplex state. For example, conformationally restricted oligonucleotide analogues that include locked nucleoside analogues (LNAs) are described in International Patent Publication No. WO 99/14226, and U.S. Pat. Nos. 5,840,728, 5,869,666, and 6,083,482, which are incorporated by reference herein in their entirety, including any figures. There is also Thio-LNA (2′S-4′C. methylen bridge) and Amino-LNA (2′NR-4′C. methylen bridge, R=H, Me), see Kumar et al. Bioorg. Med. Chem. Lett 1998, 8, 2219; and Singh et al. J. Org. Chem. 1998, 63, 10035), both of which are incorporated herein by reference in their entirety, including any drawings.

LNAs contain bicyclic nucleosides with a 2′-O-4′-C methylene bridge. This conformational and steric hindrance is believed to inhibit nuclease attack of the LNA, thereby resulting in an increased thermal stability of duplexes formed between LNA sequences and complementary DNA or RNA. To date, while LNAs have been developed as blocking agents for translation and transcription in vitro and in vivo, as sequence specific inhibitors such as PCR clamping, as well as in various antisense therapies, the capabilities of LNA have not been harnessed for the capture of SSRs. See. e.g., www.proligo.com and www.Exiqon.com.

Thus, a need still remains for an accurate, efficient method of isolating SSRs in an economical, high throughput manner that obviates the limitations and compromises attendant in current technologies. Accordingly, a need exists for a method of capturing intact SSRs from double stranded DNA, without the need for denaturation, in a method that is highly sensitive, yet not labor intensive, and be conducted with a wide diversity of nucleotide sequences.

### SUMMARY OF THE INVENTION

The present invention greatly alleviates the disadvantages of known SSR detection and isolation techniques by providing a method of capturing one or more target simple sequence repeats utilizing locked nucleic acids and strand displacement strategies.

Briefly, in a preferred embodiment, one or more modified oligonucleotide conjugates are selected in which the conjugates are constructed from at least one locked nucleic acid (“LNA”) and a linking molecule, hereinafter referred to as “LNA conjugates”. The LNA conjugates preferably are about 6-30 bases long, more preferably about 9-20 bases long (i.e., the LNA conjugate may have 9, 10, 11, 12, 13, 14, 15, 16, 17, 1.9 18, 19, or 20 bases), even more preferably about 10-15 bases long and most preferably about 12 bases long, although any number of bases in these ranges may be included in the LNA conjugate. The LNA conjugate preferably does not include any non-modified oligonucleotides, but it is possible to include some non-modified nucleic acids (or non-LNA bases), for example 1, 2, 3, 4 ,5, 6, or more bases up to all but 3 or 4 of the bases. A particular good building block to combine LNA with for DNA recognition is RNA (Acc.Chem Res 1999, 32, 301-310, Table 1) and 2′OMeRNA also may be quite good.

The modified oligonucleotide conjugate may include a peptide nucleic acid conjugates or other modified nucleic acid conjugates capable of capturing the target nucleic acid or SSR in place of LNA, for example phosphoramidates (JACS 1994, 116, 3143-3144, incorporated herein by reference in its entirety) and thiophosphoramidates (Tetrahedron Lett. 1999, 40, 7661, incorporated herein by reference in its entirety).

When incubated with a sample of nucleic acids, these LNA or other conjugates capture SSRs or other target nucleic acids by selectively binding to complementary sequences of DNA or RNA, which may be either single or double stranded. The sample of nucleic acids preferably includes a plasmid with the target nucleic acid (e.g., SSR) and a vector that equal about 2.5-4.3 kb in length, and in certain circumstances the length may be about 3.0-3.8 kb, about 3.2-3.6 kb, or about 3.4 kb. Once bound, the resulting complexes constitute hybridized duplexes containing both a targeted SSR or other target nucleic acid portion and a LNA or other conjugate portion. The sample of nucleic acids preferably includes one SSR or other target sequence, but may include 2, 3, 4, 5, 6 or more such sequences.

The captured SSRs or other captured target nucleic acids preferably are separated from the sample by using a linking source that binds to the linking molecule of the hybridized duplex and extracts the linking source with the bound duplex from the sample. In a preferred embodiment, the linking molecule is biotin and the linking source is streptavidin coated onto magnetic beads. In this embodiment, the hybridized duplex is composed of the targeted SSR or other targeted nucleic acid and an LNA or other conjugate containing a biotin linking molecule. After formation of the hybridized duplex, streptavidin coated magnetic beads are contacted with the biotin of the duplex, resulting in biotin binding with streptavidin, and the now bound duplex is isolated from the sample via magnetic separation of the beads. In one aspect of the invention, the SSRs or other targets may be detached from the LNA or other conjugates and the magnetic beads by treatment with an alkaline buffer. In another aspect, the present invention discloses a sequence specific kit for capturing target simple sequence repeats or other targets that comprises the above-referenced LNA-SSR hybridized duplex or other similar duplex, a linking source, and a means for separating the hybridized duplexes from a sample of nucleic acids.

In the present disclosure, an “oligonucleotide” has its usual term in molecular biology. It refers to a polymer of nucleobases that can hybridize by Watson-Crick base pairing to a complementary sequence. Nucleobases comprise a base, which is purine, pyrimidine, or a derivative or analog thereof.

References herein to a “portion” of a DNA or RNA chain, or of a gene, mean a linear chain that has a nucleotide sequence which is the same as a sequential subset of the sequence of the chain to which the portion refers.

In the present disclosure, the term “complementary” has its usual meaning from molecular biology. Two nucleotide sequences or strands are complementary if they have sequences which would allow base pairing (Watson-Crick or Hoogsteen) according to the usual pairing rules. This does not require that the strands would necessarily base pair at every nucleotide; two sequences can still be complementary with a low level (e.g., about 1-3%) of base mismatch such as that created by deletion, addition, or substitution of one or a few (e.g., up to 5 in a linear chain of 25 bases) nucleotides, or a combination of such changes.

In the present disclosure, “hybridize” has its usual meaning from molecular biology. It refers to the formation of a base-paired interaction between nucleotide polymers. The presence of base pairing implies that a fraction of the nucleotides (e.g., at least 80%) in each of two nucleotide sequences are complementary to the other according to the usual base pairing rules. The exact fraction of the nucleotides which must be complementary in order to obtain stable hybridization will vary with a number of factors, including nucleotide sequence, salt concentration of the solution, temperature, and pH.

In the present disclosure, “duplex” has its usual meaning in molecular biology and refers to strands of nucleic acids wherein each base of a first strand of the duplex corresponds to a base of a second strand of the duplex according to the scheme in which uracil or thymine and adenine correspond and cytosine and guanine correspond. Anti-parallel duplex strands having these correspondences are said to be Watson-Crick paired. Duplex nucleic acids can be of two major types, ribonucleic acids and deoxyribonucleic acids. Each ribonucleotide has an equivalent deoxyribonucleotide, e.g., adenosine and deoxyadenosine, cytidine and deoxycytidine, guanosine and deoxyguanosine, uridine and thymidine.

References herein to the term “correspond,” as in, for example, cDNA which “corresponds” to mRNA, means that at least a portion of one nucleic acid molecule is either complementary or homologous to a second nucleic acid molecule. Thus, a cDNA molecule may correspond to the mRNA molecule where the mRNA molecule was used as a template for reverse transcription to produce the cDNA molecule. Similarly, a genomic sequence of a gene may correspond to a cDNA sequence where portions of the genomic sequence are homologous or complementary to the cDNA sequence.

Because locked nucleic acids may be derivatives of any nucleobase, the limitations placed on triplex affinity capture, for example, the necessity of homopurine or homopyrimidine rich residues in the target sequences, are absent from the present invention. Advantageously, conjugates composed of LNAs may selectively isolate and bind with a wide diversity of nucleotidic sequences, unhampered by base limitations. For example, the present invention is capable of isolating SSRs composed of heteropurine or heteropyrimidine bases that are currently not detectable using a triplex affinity capture method.

Advantageously, LNAs form more stable duplexes than their DNA or RNA counterparts. As such, the ability of LNAs to form stable duplexes under conditions, such as low ionic strength or in buffers containing strong chaotropic agents, i.e., conditions in which RNA-DNA or DNA-DNA duplexes are unattainable, manifests itself in LNAs' increased hybridization affinities for target SSRs.

The enhanced stability of LNAs is particularly beneficial when isolating very short sequences such as SSRs. Higher ionic strength conditions induce intrastrand folding of target DNA or RNA sequences. By lowering the salt conditions, decreased intrastrand folding results, thereby exposing more of the targeted sequence to hybridization. Because of SSRs' low frequency of occurrence in genomic strands as well as the shortness of the sequences, accessibility of the target strand is essential.

As another advantage related to the accessibility of target sequences, LNA sequences are particularly useful where the target sequence is difficult or impossible to access by unmodified oligonucleotide sequences due to the rapid formation of stable intramolecular structures such as those occurring in rRNA, tRNA, snRNA, and scRNA. LNAs present a distinct advantage over unmodified oligonucleotides in that the steric bulkiness and rigidity of the bicyclic component of LNAs hinder such intramolecular folding, thereby increasing the accessibility of infrequently occurring and short sequenced SSRs.

Because of the enhanced stability and thus, affinity, of LNA complexes, single mismatches in LNA duplexes cause considerable drops in thermal stability and result in a heightened ability to discriminate single base pair differences compared to conventional RNA-DNA or DNA-DNA duplexes.

As a further advantage, LNA sequences can capture target SSR sequences, possibly via strand displacement in which the LNA sequence selectively binds to its complementary target sequence in double stranded DNA or RNA such that the second strand of the double strand is displaced. Unlike colony or plaque hybridization, which frequently require prior denaturation steps, the present invention is able to detect and isolate intact double stranded DNA. The strand displacement mechanism of the present invention also obviates the labor intensive, multi-step washing procedures required of colony and plaque hybridization techniques.

Another advantage of the present invention stems from the availability of a linking molecule. The linking molecule may be attached to LNA at either end of the modified oligonucleotide, at one or more internal positions, or via spacers attached to the 5′ or 3′ end according to the methods disclosed in PCT WO 99/14226, which is incorporated by reference herein in its entirety, including any figures.

### BRIEF DESCRIPTION OF THE DRAWINGS

These and other features and advantages of the present invention will be appreciated from the following detailed description, along with the accompanying figure in which like reference numerals identify like elements throughout and wherein:

FIG. 1 shows a schematic diagram of a method of isolating simple sequence repeats made in accordance with the present invention.

This figure is a schematic representation for purposes of illustration and does not necessarily depict the actual relative sizes or locations of the elements shown.

### DETAILED DESCRIPTION OF THE DRAWINGS

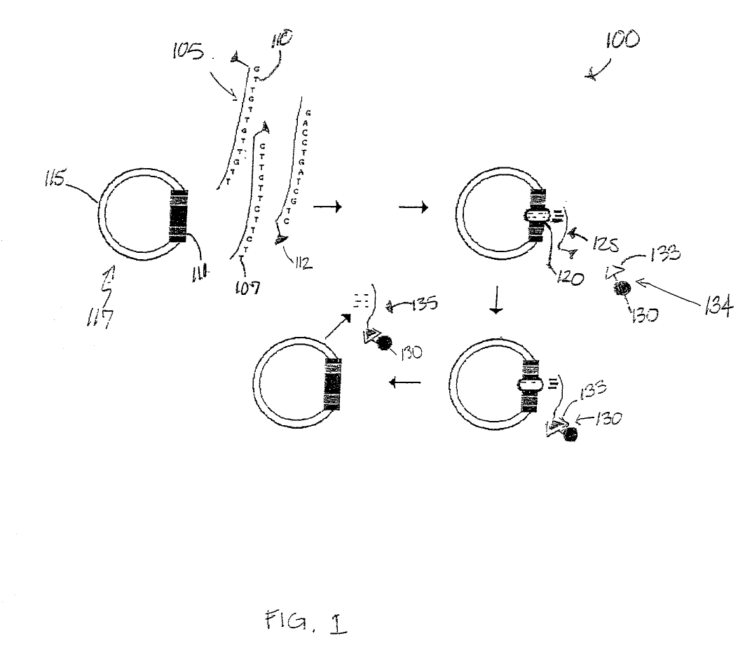

In the following paragraphs, the present invention will be described in detail by way of example with reference to FIG. 1. Throughout this description, the preferred embodiments and examples shown should not be considered as limiting the scope of the present invention.

FIG. 1 shows a method 100 of detecting, isolating, and separating simple sequence repeats (SSRs) from a nucleic acid sample. This method may be automated or conducted manually on either a laboratory benchtop or industrial scale. In the disclosed example, a modified oligonucleotide conjugate 105 (“LNA conjugate”) is selected wherein conjugate 105 contains an LNA nucleotide sequence complementary to a target within 111 and LNA conjugate 105 is composed of at least one locked nucleic acid (LNA) 107 and a linking molecule 112. The number, type and combination of LNAs 107 that comprise LNA oligonucleotide sequence 110 will depend on the nucleotide sequence of a target SSR 120 contained in 111.

Generally, an LNA oligonucleotide sequence 110 will be chosen that is complementary to the target SSR 120 to be captured. For example, if the target SSR 120 has a nucleotide sequence of 5′-(CA)₆-3′, a preferred LNA oligonucleotide sequence 110 would be 3′-(GT)₆-5′. (Alternatively, the complementary sequence in the target dsDNA could be targeted by a complementary LNA sequence). However, because strand recognition permits some degree of mismatch, each LNA oligonucleotide 107 need not correspond exactly to each SSR nucleotide according to its Watson-Crick pairing. Any LNA oligonucleotide sequence 110 which will selectively bind to a target SSR sequence 120 is contemplated within the present invention. LNA oligonucleotides sequences 110 may be obtained commercially from Proligo, LLC (Boulder, Colo.). Alternatively, LNA oligonucleotide sequences 110 may be synthesized according to the methods described in PCT WO 99/14226, or by any other methods know to those skilled in the art of synthesizing modified oligonucleotide sequences.

A linking molecule 112 is attached to LNA oligonucleotide sequence 110 by any method known in the art. For example, methods of attaching biotin to an LNA olignonucleotide sequence 110 at its 3′ or 5′ ends are described in PCT WO 99/14226. In a preferred embodiment, the linking molecule 112 is biotin. Other linking molecules 112 such as antibodies, immunoglobulins, or carbohydrates may also be used. Alternatively, linking molecule 112 may be any molecule that may be recognized by a linking source 134 and used to extract targeted SSR 120 from a sample. Preferably, linking molecule 112 should have a high capacity of molecular recognition for linking source 134 and a high capacity to bind to linking source 134. Linking molecules 112 with such an affinity to linking source 134 are conventional and therefore are not described in detail herein. Techniques for preparing and utilizing such systems are well-known in the literature and are exemplified in, for example, the publication Tijssen, P., Laboratory Techniques in Biochemistry and Molecular Biology Practice and Theories of Enzyme Immunoassays, eds. Burdon and Knippenberg, N.Y., Elsevier Press, (1988).

The LNA conjugates 105 are incubated with a sample of nucleic acids 117 containing the target SSR 120. Incubation conditions are dependent upon the LNA oligonucleotide sequence 110 utilized in accordance with this invention. For example, temperature conditions are specific to the type of LNA oligonucleotide sequence 110 and preferably is about 80° C.

A wide variety of samples 117 are contemplated within the present invention. For example, the sample 117 may be obtained from genomic libraries, DNA sequences, genomic DNA, RNA sequences, plasmids, double stranded DNA, double stranded DNA plasmid libraries, and single stranded DNA. Preferably, the sample is obtained from a plasmid library, and more preferably, a circular plasmid library, and even more preferably, from 3.5 kb double stranded clones (plasmid and insert). Alternatively, the present invention contemplates obtaining sample 117 from any source in which SSRs are present. In a preferred embodiment, sample 117 contains target SSRs 120 from a genomic library in pBluescript KS-/sacB construct. Multiple and different SSRs 120 may be found in any sample 117. Target SSR 120 may be of any length, but preferably comprise between 1 and 4 base pair repeats.

Once incubated together, LNA conjugate 105 and target SSR 120 form hybridized duplex 125. Because of the higher annealing temperature and higher thermal stability of LNA duplexes than corresponding DNA or RNA duplexes, LNA conjugate 105 invades double-stranded DNA and aligns itself with the complementary sequence of target SSR 120, thereby forming an “A” helix, reminiscent of a DNA-RNA double helix. Beneficially, this enables the present invention to capture SSRs 120 from double-stranded DNA, and represents an advantage over current technologies which are not able to isolate SSRs from double stranded DNA without denaturation.

After hybridized duplex 125 is formed, duplex 125 is contacted with a linking source 134 to form a linked complex 135 comprised of duplex 125 bound, either covalently or uncovalently, with linking source 134 through linking molecule 112. In a preferred embodiment, linking source 134 comprises magnetic beads 130 and streptavidin molecules 133 attached to magnetic beads 130. In the disclosed example, contact of linking source 134 with duplex 125 may be accomplished by introducing the streptavidin coated beads 130 into sample 117 containing hybridized duplex 125 and shake the solution to create a suspension of magnetic beads 130, thereby maximizing contact with hybridized duplex 125. Alternatively, hybridize duplex 125 may be stirred or vortexed with linking source 134; duplex 125 may be passed down a column or fluidized bed of linking source 134, or contacted with linking source 134 with no movement at all, allowing equilibrium conditions to attach duplex 125 to linking source 134.

The time period for contact between duplex 125 and linking source 134 is dependent upon the equilibrium conditions necessary for linking molecule 112 to attach to linking source 134, and is easily ascertainable by practitioners of the art. For example, a smaller time period may be needed to attach linking molecule 112 for duplexes 125 that are vigorously shaken with linking source 134, than if linking molecule 112 was introduced into solution with duplex 125 without shaking. Similarly, the time period for attachment may be less or more if linking molecule 112 was an antibody and linking source 134 was a corresponding antigen coated onto magnetic beads, than if linking molecule 112 and linking source 134 were biotin and streptavidin, respectively.

The present invention contemplates using any molecule 133 capable of recognizing linking molecule 112 that is also attachable onto a solid support 130, as a viable linking source 134. For example, the linking source may be composed of avidin, which recognizes biotin; antibodies that recognize their corresponding antigens; protein A and its corresponding immunoglobulin; or lectin that recognizes corresponding carbohydrates. Solid support 130 may be any solid material that is capable of forming a bond, either covalently or uncovalently, with molecule 133 without preventing binding between molecule 133 and linking molecule 125. As examples, solid support 130 may include agarose, cellulose, nitrocellulose, cross-linked dextrose, silicon, silica, nylon, glass, metallic and magnetic compositions and numerous plastics. Preferred structures are particles or beads but tubes, disks, and microplates are also acceptable. For example, a preferred embodiment uses magnetic beads as solid support 10.

Following incubation and attachment of linking molecule 112 with linking source 134, the resulting linking complex 135 is separated from sample 117. The method of separation will depend upon the type of linking source 134 and linking molecule 112 selected. In a preferred embodiment, an applied magnetic field such as a magnet separates linking complex 135 from sample 117 and any concomitant reaction medium when linking molecule 112 is biotin and linking source 134 is streptavidin coated magnetic beads. Alternatively, sample 117 and any reaction solution may be decanted from linking complex 135. Column or bed washings with an appropriate buffer, centrifugation, and filtering are also contemplated within the present invention.

In a preferred embodiment, treatment of linking complex 135 with a slightly alkaline buffer disrupts the hydrogen bonds between targeted SSR 120 and LNA conjugate 105, thereby disassociating the targeted SSR 120 from LNA conjugate 105 and linking source 134. Preferably, the pH of the buffer is between 8.0 and 10.0 and more preferably between 9.0-9.5. The time period and temperature conditions of the treatment are dependent upon the amount of SSR 120 isolated and the specific type of SSR 120, and is readily ascertainable by practitioners in the art. Once disassociated, targeted SSR 120 may be recovered from the buffer solution by conventional methods such as phenyl/chloroform extraction (1:1 (vol/vol)) and ethanol precipitation or electrophoresis.

Once isolated, subsequent bacterial transformation produces very pure and substantial quantities of SSRs for a wide variety of uses such as genetic markers.

In another aspect of this invention, a hybridized duplex 125 is described. The duplex comprises a locked nucleic acid portion and a simple sequence repeat portion. The locked nucleic acid portion contains any LNA conjugate 105 wherein LNA conjugate 105 contains LNA nucleotide sequence 110 composed of at least one LNA 107 and linking molecule 112. The number, type and combination of LNAs 107 that comprise LNA oligonucleotide sequence 110 will depend on the nucleotide sequence of target SSR 120. Typically, LNA oligonucleotide sequence 110 is chosen to be complementary to target SSR 120 to be captured, and is described above. Target SSR 120 may be of any length, but preferably comprise between 1 and 10 base pair sequences, and more preferably between 1 and 4 base pair sequences. In one aspect of the invention, the hybridized duplex also comprises a linking source 134.

In yet another aspect of the invention, a kit for capturing target SSR 120 is also described. This kit contains one or more LNA oligonucleotide conjugates 105, hybridized duplex 125 comprised of a target SSR portion and a LNA conjugate portion, linking source 134, and a means for separating hybridized duplexes 125 from any sample of nucleic acids 117 that contain target SSR 120. Preferably, the means for separating hybridized duplex includes an alkaline buffer, which disassociates target SSR 120 from LNA conjugate 105 and linking source 134. More preferably, the alkaline buffer has a pH between 9 and 10.

### EXAMPLE

One aspect of this invention, namely a method of capturing target simple sequence repeats, was demonstrated using a plasmid pBluescript KS-/sacB containing a known C-A repeat. 5′ biotinylated LNA oligonucleotide (GT)₆ was obtained from Proligo, LLC (Boulder, Colo.) (referred hereinafter as “LNA oligo”). Streptavidin-coated magnetic beads (Dynabeads Streptavidin M-280) and a magnetic particle concentrator (Dynal MPC-?) were obtained from Dynal, Inc. (Great Neck, N.Y.). Streptavidin-coated magnetic beads were washed and resuspended with hybridization buffer prior to use.

1 μg plasmid was incubated with 75 ng LNA oligo in 60 μl hybridization buffer (100 mM NaCl, 10 mM Na₂PO₄, pH 7.0) for 50 minutes at 80° C. 60 μl washed Dynabeads were incubated with the LNA oligo/plasmid hybridization medium for 80 minutes in a 50° C. water bath. Shaking of the water bath ensued every 10 minutes to maintain a suspension of the beads.

After removal from the water bath, the hybridization medium was centrifuged and Dynabeads separated using a Dynal magnet. Supernatant was removed and the beads washed 8 times with 500 μl of hybridization buffer. 150 μl of disassociation buffer (1.0 M Tric-Cl, pH 9.0, 0.5 mM EDTA) was added to the bead mixture with stirring. The resulting mixture was incubated at 90° C. for 20 minutes.

After centrifugation, the supernatant was retained and the beads discarded. 200 μl of ddH₂O (pH 5.0), 35 μl of 3 M NaOAc (pH 5.2), and 875 μl of 100% EtOH was added to the supernatant. The resulting solution was incubated at −20° C. for at least 12 hours. The resulting mixture was centrifuged at 14,000 rpm for 30 minutes. After decanting the supernatant, the precipitate was washed with 500 μl of ice-cooled 70% EtOH and centrifuged for 20 minutes. After decanting the supernatant, the resulting precipitate was dried and 100 μl 1× TE (pH 8.0) was added. The resulting mixture was purified using a Qiagen PCR purification kit.

Life Technologies DH12s electrocompetent cells were transformed with 1 μl DNA to 40 μl cells using a BioRad Gene Pulser II (25 μF, 200 Ω, 1.8 kV). Upon addition of SOC broth, the transformation mixture was shaken gently for 1 h at 37° C. 75 μl of transformation mixture were plated onto LB amp plates containing 5% sucrose. Upon room temperature incubation for at least 12 h, colonies were collected into LB plates with 8% glycerol. After room temperature incubation for at least 12 h, the resulting colonies were stored at −80° C. for sequencing.

One skilled in the art will appreciate that the present invention can be practiced by other than the preferred embodiments which are presented in this description for purposes of illustration and not of limitation, and the present invention is limited only by the claims that follow. It is noted that equivalents for the particular embodiments discussed in this description are also within the scope of the present invention and that preferred embodiments or features of one aspect of the invention can be incorporated into any of the other aspects of the present invention.

What is claimed is:

1. An in vitro method of capturing one or more target simple sequence repeats, wherein the method comprises the steps of:
providing one or more modified oligonucleotide conjugates, wherein each of the modified oligonucleotide conjugates comprises at least one locked nucleic acid and a linking molecule;
incubating a sample of nucleic acids with the modified oligonucleotide conjugates, thereby forming one or more hybridized duplexes, wherein each duplex comprises a target simple sequence repeat portion and a modified oligonucleotide conjugate portion;
contacting substantially all of the hybridized duplexes with a linking source, such that the linking molecule of each duplex that contacts the linking source forms a bond with the linking source; and
separating substantially all of the hybridized duplexes from the sample of nucleic acids by extracting the linking source from the sample.

2. The method of claim 1, wherein the sample of nucleic acids contains one or more nucleic acid molecules having a nucleotide sequence which comprises one or more target simple sequence repeats.

3. The method of claim 1, wherein the captured simple sequence repeat portion comprises 1, 2, 3, or 4 base repeats.

4. The method of claim 1, further comprising the step of disassociating the targeted simple sequence repeat portion of each of the hybridized duplexes from the linking source and modified oligonucleotide conjugate.

5. The method of claim 4, wherein the disassociating step further comprises incubating the hybridized duplexes with an alkaline buffer, such that the targeted simple sequence repeat portion is disassociated from the modified oligonucleotide conjugate and the linking source.

6. The method of claim 5, wherein the alkaline buffer has a pH between 9 and 10.

7. The method of claim 1, wherein the incubating step comprises using reaction conditions at which substantially all of the targeted simple sequence repeats form a strand displacing “A” helix with the modified oligonucleotide conjugates.

8. The method of claim 1, wherein the method forms a new library enriched in the targeted simple sequence repeat or repeats.

9. A library enriched with targeted simple sequence repeats, formed by the method of claim 1.

10. The library according to claim 9, wherein the targeted simple sequence repeats comprise 1, 2, 3, or 4 base repeats.

11. The method of claim 1, wherein the simple sequence repeat portion of the hybridized duplex is a portion of the insert in the 3.5 kb clone.

12. The method of claim 1, further comprising the step of obtaining the sample of simple sequence repeats from a plasmid library.

13. The method of claim 12, wherein at least one of the one or more plasmids is a circular plasmid.

14. The method of claim 1, further comprising the step of obtaining the sample of simple sequence repeats from a double stranded DNA plasmid library.

15. The method of claim 1, further comprising the step of obtaining the sample of simple sequence repeats from DNA sequences.

16. The method of claim 1, further comprising the step of obtaining the sample of simple sequence repeats from genomic DNA.

17. The method of claim 1, further comprising the step of obtaining the sample of simple sequence repeats from RNA sequences.

18. The method of claim 1, wherein the modified oligonucleotides comprise nucleotide sequences that are complementary to the target simple sequence repeats.

19. The method of claim 1, wherein the linking molecule comprises biotin bound to a 5′ end of the modified oligonucleotide.

20. The method of claim 1, wherein the linking molecule comprises an antibody, biotin, immunoglobulin, or carbohydrate.

21. The method of claim 1, wherein the linking source comprises streptavidin-coated beads.

22. The method of claim 1, wherein the linking source comprises an antigen, avidin, protein A, or lectin.

23. The method of claim 1, wherein the separating step comprises using a magnet to extract the linking source and hybridized duplexes from the sample of simple sequence repeats.

24. An in vitro method of capturing one or more target simple sequence repeats, wherein the method comprises the steps of:
providing one or more modified oligonucleotide conjugates, wherein each of the modified oligonucleotide conjugates comprises at least one locked nucleic acid and a linking molecule;
incubating a sample of nucleic acids with the modified oligonucleotide conjugates, thereby forming one or more hybridized duplexes, wherein each duplex comprises a target simple sequence repeat portion and a modified oligonucleotide conjugate portion:
binding the linking molecule biotin on the modified oligonucleotides to streptavidin on coated magnetic beads, such that the magnetic beads are linked to the hybridization duplexes;
separating the hybridized duplexes from other materials with a magnet;
washing the hybridized duplexes;
incubating the hybridized duplexes with buffer of pH of about 9.5 such that the targeted simple sequence repeat dissociates from the modified oligonucleotide conjugate and the magnetic bead;
transforming the simple sequence repeats in E coli; and
sequencing the transformed simple sequence repeats.

25. A hybridized duplex comprising:
a locked nucleic acid portion, and
a simple sequence repeat portion.

26. The hybridized duplex of claim 25, further comprising a linking source.

27. A strand displacement method of capturing target nucleic acids, wherein the improvement comprises using one or more target nucleic acid molecules having a nucleotide sequence which comprises one or more simple sequence repeats.

28. A kit for capturing target simple sequence repeats, comprising:
one or more modified oligonucleotide conjugates, wherein each of the modified oligonucleotide conjugates comprises at least one locked nucleic acid and a linking molecule.
a hybridized duplex comprised of a simple sequence repeat portion and a modified oligonucleotide conjugate portion;
a linking source, wherein the linking source is attachable to the linking molecule of the modified oligonucleotide conjugate; and
a means for separating the hybridized duplexes from a sample of nucleic acids.

29. The kit of claim 28, further comprising an alkaline buffer.

30. The kit of claim 28, wherein the buffer has a pH between 9 and 10.
